(19) United States
(12) Patent Application Publication
(10) Pub. No.: US 2026/0166149 A1
(43) Pub. Date: Jun. 18, 2026

(54) NEAR-INFRARED RESPONSIVE BILIRUBIN COMPOSITE NANOPARTICLE-LOADED PERIODONTAL MICRONEEDLE AND PREPARATION METHOD THEREOF

(71) Applicant: STOMATOLOGICAL HOSPITAL OF CHONGQING MEDICAL UNIVERSITY, Chongqing (CN)

(72) Inventors: Xiang GAO, Chongqing (CN); Yue WANG, Chongqing (CN); Wenliang YU, Chongqing (CN); Jinlin SONG, Chongqing (CN)

(21) Appl. No.: 18/845,619

(22) PCT Filed: Dec. 26, 2023
(86) PCT No.: PCT/CN2023/142075

§ 371 Date: Sep. 10, 2024

(30) Foreign Application Priority Data
Apr. 4, 2023 (CN) 202310352354.1

(51) Int. Cl. A61K 45/06; A61K 9/00; A61K 9/14; A61K 38/00; A61K 41/00

(52) U.S. Cl. CPC A61K 45/06; A61K 9/0019; A61K 9/14; A61K 38/00; A61K 41/0071

(57) ABSTRACT

A near-infrared responsive bilirubin composite nanoparticle-loaded periodontal microneedle and a preparation method thereof are provided. A near-infrared responsive bilirubin composite nanoparticle includes a bilirubin-gelatin composite nanoparticle, wherein a surface of the bilirubin-gelatin composite nanoparticle is coated with a macrophage cell membrane and an organic-metal coordination supramolecular network coating in sequence; an outer surface of the near-infrared responsive bilirubin composite nanoparticle is modified with 1,2-distearoyl-sn-glycero-3-phosphoethanolamine-N-[folate(polyethylene glycol)] (DSPE-PEG-FA); in the bilirubin-gelatin composite nanoparticle, bilirubin and gelatin are covalently bonded via an amide bond; and in the organic-metal coordination supramolecular network coating, an organic ligand is anthocyanin, and a coordinating metal ion is ferric ion.

### CROSS-REFERENCE TO RELATED APPLICATION

The present application is a national stage application of International Patent Application No. PCT/CN2023/142075, filed on Dec. 26, 2023, which claims priority to Chinese Patent Application No. 202310352354.1 filed with the China National Intellectual Property Administration (CNIPA) on Apr. 4, 2023 and entitled “NEAR-INFRARED RESPONSIVE BILIRUBIN COMPOSITE NANOPARTICLE-LOADED PERIODONTAL MICRONEEDLE AND PREPARATION METHOD THEREOF”. The disclosure of the two applications is incorporated by references herein in their entireties as part of the present application.

### TECHNICAL FIELD

The present disclosure relates to the technical field of composites, and in particular relates to a near-infrared responsive bilirubin composite nanoparticle-loaded periodontal microneedle and a preparation method thereof.

### BACKGROUND

Periodontitis, as a worldwide prevalent chronic inflammatory disease, could continuously destroy the periodontal supporting tissue, and is the main cause of tooth loosening and loss and a potential factor that causes systemic inflammation. Currently, periodontitis has become the sixth most common disease in human beings. According to statistics, approximately 796 million adults worldwide are suffered from periodontal health problems, bringing a huge economic burden to patients and the medical system. Plaque microorganisms are the initiating factor of periodontitis. At present, the main treatment method for periodontitis is to control the development of this disease by removing bacterial plaque. However, the main cause of periodontal destruction—a dysregulated immune-inflammatory response of the host—has not been improved, and meanwhile tissue regeneration ability is impaired, so that the regeneration effect of the periodontal tissues is not ideal.

With the deepening of relevant research in recent years, the damage to periodontal tissues caused by imbalance of host immune homeostasis in the pathological environment of periodontitis has been apparently clear. The underlying mechanism involves a large accumulation of reactive oxygen species (ROS) caused by mitochondrial and endoplasmic reticulum dysfunction, and the release of a large amount of pro-inflammatory factors caused by over-activated immune response, which thus ultimately affects the repair of periodontal tissues, resulting in a decrease in the efficiency and quality of new bone formation as well as active bone destruction and resorption.

Macrophages play a key role in the regulation of host immune homeostasis. Normally, a large number of resident unactivated macrophages (M_{φ}) from the circulation system and tissues are recruited and activated to differentiate into different phenotypes-pro-inflammatory macrophages (M1) and anti-inflammatory macrophages (M2), which participate in identifying and removing pathogens and conducting tissue regeneration and repair, respectively. However, in the periodontal tissues at the inflammatory site, a large accumulation of ROS due to dysfunction of mitochondria and endoplasmic reticulum leads to the destruction of M1/M2 macrophage dynamic balance. Macrophages are unable to repolarize from a pro-inflammatory phenotype to an anti-inflammatory phenotype, resulting in the secretion of pro-inflammatory cytokines in a large amount. These pro-inflammatory cytokines promote osteoclast differentiation, thereby eventually causing alveolar bone resorption and hindering periodontal regeneration and repair.

### SUMMARY

In view of this, an object of the present disclosure is to provide a near-infrared responsive bilirubin composite nanoparticle-loaded periodontal microneedle and a preparation method thereof. In the present disclosure, the near-infrared responsive bilirubin composite nanoparticle could reshape the immune homeostasis of periodontitis and have a desirable therapeutic effect on the periodontitis.

To achieve the above object, the present disclosure provides the following technical solutions:

The present disclosure provides a near-infrared responsive bilirubin composite nanoparticle, including a bilirubin-gelatin composite nanoparticle, wherein
a surface of the bilirubin-gelatin composite nanoparticle is coated with a macrophage cell membrane and an organic-metal coordination supramolecular network coating in sequence; and an outer surface of the near-infrared responsive bilirubin composite nanoparticle is modified with 1,2-distearoyl-sn-glycero-3-phosphoethanolamine-N-[folate(polyethylene glycol)] (DSPE-PEG-FA);
in the bilirubin-gelatin composite nanoparticle, bilirubin (BR) and gelatin (GA) are covalently bonded via an amide bond; and
in the organic-metal coordination supramolecular network coating, an organic ligand is anthocyanin, and a coordinating metal ion is ferric ion.

In some embodiments, the bilirubin-gelatin composite nanoparticle has a particle size of 100 nm to 300 nm; and
the near-infrared responsive bilirubin composite nanoparticle has a particle size of 100 nm to 500 nm.

The present disclosure further provides a method for preparing the near-infrared responsive bilirubin composite nanoparticle, including the following steps:
mixing the bilirubin, the gelatin, a carboxyl activator, and an organic solvent, and subjecting a resulting mixture to amidation reaction, to obtain a bilirubin-gelatin composite;
mixing an aqueous dispersion of the bilirubin-gelatin composite with an organic solvent, and subjecting a resulting mixture to self-assembly, to obtain a bilirubin-gelatin composite nanoparticle;
subjecting the bilirubin-gelatin composite nanoparticle and the macrophage cell membrane to co-extrusion, to obtain a cell membrane-coated bilirubin-gelatin composite nanoparticle;
mixing an aqueous dispersion of the cell membrane-coated bilirubin-gelatin composite nanoparticle, the anthocyanin, a soluble ferric ion source, and water, and adjusting a pH value of a resulting mixed solution to 7.5 to 8, to obtain an organic-metal coordination supramolecular network coating-coated bilirubin-gelatin composite nanoparticle; and
mixing an aqueous dispersion of the organic-metal coordination supramolecular network coating-coated bilirubin-gelatin composite nanoparticle with the DSPE-PEG-FA, and subjecting a resulting mixture to incubation, to obtain the near-infrared responsive bilirubin composite nanoparticle.

In some embodiments, a mass ratio of the bilirubin to the gelatin is in a range of 1:10 to 1: 15; and
the amidation reaction is conducted at room temperature for 8 h to 10 h.

In some embodiments, a mass ratio of the bilirubin-gelatin composite nanoparticle to the macrophage cell membrane is in a range of 1:1 to 2:1.

In some embodiments, a mass ratio of the anthocyanin, the soluble ferric ion source, and the cell membrane-coated bilirubin-gelatin composite nanoparticle is in a range of 5:1:(4-5).

In some embodiments, a mass ratio of the organic-metal coordination supramolecular network coating-coated bilirubin-gelatin composite nanoparticle to the DSPE-PEG-FA is in a range of 4:1 to 10:1; and
the incubation is conducted at a temperature of 35° C. to 37° C. for 0.5 h to 2 h.

The present disclosure further provides use of the near-infrared responsive bilirubin composite nanoparticle as described above in preparation of a drug for treating periodontitis.

The present disclosure further provides a near-infrared responsive bilirubin composite nanoparticle-loaded microneedle for periodontitis, including a microneedle tip and a microneedle base of a drug loading microneedle, wherein raw materials for preparing the microneedle tip include gelatin methacryloyl (GelMA), a photoinitiator, and the near-infrared responsive bilirubin composite nanoparticle as described above.

In some embodiments, a mass ratio of the GelMA, the photoinitiator, and the near-infrared responsive bilirubin composite nanoparticle is in a range of 200:5:(8-10).

The present disclosure provides a near-infrared responsive bilirubin composite nanoparticle, including a bilirubin-gelatin composite nanoparticle, wherein a surface of the bilirubin-gelatin composite nanoparticle is coated with a macrophage cell membrane and an organic-metal coordination supramolecular network coating in sequence; and an outer surface of the near-infrared responsive bilirubin composite nanoparticle is modified with the DSPE-PEG-FA; in the bilirubin-gelatin composite, bilirubin and gelatin are covalently bonded via an amide bond; and in the organic-metal coordination supramolecular network coating, an organic ligand is anthocyanin, and a coordinating metal ion is ferric ion. In the present disclosure, the bilirubin-gelatin composite nanoparticle shows desirable antioxidant activity and promotes the M2 repolarization of macrophages by scavenging ROS. A surface of the bilirubin-gelatin composite nanoparticle is coated with an organic-metal coordination supramolecular network coating. The organic-metal coordination supramolecular network coating is obtained by coordination of anthocyanin (cyanidin-3-O-β-glucoside, C3G) and ferric ions, which form the organic-metal coordination supramolecular network coating through coordination bonds. The coating has both the properties of anthocyanin and iron ions, and exhibits anti-inflammatory and antioxidant therapeutic effects. Meanwhile, the coating has photothermal properties and could convert near-infrared (NIR) light energy into heat energy to induce apoptosis of M1 macrophages. Accordingly, the near-infrared responsive bilirubin composite nanoparticle has dual functions in stimulating the apoptosis of M1 macrophages with photothermal effect and scavenging the ROS to induce M2 polarization of M1 macrophages, thereby reshaping the immune homeostasis of periodontitis. The DSPE-PEG-FA could pass through pores in the organic-metal coordination supramolecular network coating and insert into a phospholipid bilayer of the macrophage cell membrane, thereby modifying an outer surface of the composite nanoparticle. Since folate (FA) could specifically bind to FA receptors on the surface of M1 macrophages, it endows the nanoparticles with good MI macrophage targeting properties and improves bioavailability of the nanoparticles.

The present disclosure further provides a method for preparing the near-infrared responsive bilirubin composite nanoparticle as described above. The method is simple to operate and easy to realize industrial mass production.

The present disclosure further provides a near-infrared responsive bilirubin composite nanoparticle-loaded microneedle for periodontitis, including a microneedle tip and a microneedle base of a drug loading microneedle, wherein raw materials for preparing the microneedle tip include GelMA, a photoinitiator, and the near-infrared responsive bilirubin composite nanoparticle as described above. Due to the complex and changeable environment of oral periodontal anatomy, which is affected by multiple factors such as chewing, saliva secretion, and bacterial infection, it is difficult to achieve long-term and stable drug delivery. The near-infrared responsive bilirubin composite nanoparticle-loaded microneedle for periodontitis according to the present disclosure has desirable mechanical properties and could penetrate deep periodontal tissues to achieve efficient delivery. Moreover, the microneedle could reshape the immune homeostasis of periodontitis by dual functions in regulating and stimulating the apoptosis of M1 macrophages and scavenging ROS to induce the repolarization of M1 macrophages, thus ultimately promoting the regeneration and repair of periodontal tissues.

### BRIEF DESCRIPTION OF THE DRAWINGS

FIG. 1 shows flowcharts of the preparation of the near-infrared responsive bilirubin composite nanoparticle and a drug-loaded microneedle for periodontitis.

FIG. 2A to FIG. 2C show fluorescence co-localization images of BRNP (FIG. 2A), membrane (FIG. 2B), and mBRNP (FIG. 2C).

FIG. 3A and FIG. 3B show transmission electron microscopy (TEM) images of BRNP (FIG. 3A) and mBRNP@C3G-Fe³⁺ (FIG. 3B).

FIG. 4 shows particle size distributions of BRNP, mBRNP, and mBRNP@C3G-Fe³⁺.

FIG. 5 shows particle sizes of mBRNP@C3G-Fe³⁺ and FA-mBRNP@C3G-Fe³⁺.

FIG. 6 shows potentials of mBRNP@C3G-Fe³⁺ and FA-mBRNP@C3G-Fe³⁺.

FIG. 7A to FIG. 7F show phagocytosis results of FA-mBRNP@C3G-Fe³⁺ by different cells, in which, FIG. 7A shows a phagocytosis result of FA-mBRNP@C3G-Fe³⁺ by HGF when being stained with DAPI (4′,6-diamidino-2-phenylindole); FIG. 7B shows a phagocytosis result of FA-mBRNP@C3G-Fe3+by M_{φ} when being stained with DAPI; FIG. 7C shows a phagocytosis result of FA-mBRNP@C3G-Fe³⁺ by M₁ when being stained with DAPI; FIG. 7D shows a fluorescence microscope observation result of FA-mBRNP@C3G-Fe³⁺ after co-culture with HGF; FIG. 7E shows a fluorescence microscope observation result of FA-mBRNP@C3G-Fe³⁺ after co-culture with M_{φ}; and FIG. 7F shows a fluorescence microscope observation result of FA-mBRNP@C3G-Fe³⁺ after co-culture with M₁.

FIG. 8 shows temperature-time change curves in photothermal imaging.

FIG. 9A shows photothermal performance of mBRNP@C3G-Fe³⁺); and FIG. 9B shows the corresponding linear relationship between-Ln (θ) and time (min).

FIG. 10 shows photothermal-induced apoptosis of M1 macrophages.

FIG. 11A to FIG. 11H show detection results of apoptosis using Annexin V-FITC apoptosis detection kit, in which, FIG. 11A shows a detection result of control group without NIR irradiation; FIG. 11B shows shows a detection result in bright field of control group without NIR irradiation; FIG. 11C shows a detection result of control group with NIR irradiation; FIG. 11D shows a detection result in bright field of control group with NIR irradiation; FIG. 11E shows a detection result of experimental group (FA-mBRNP@C3G-Fe³⁺) without NIR irradiation; FIG. 11F shows a detection result in bright field of experimental group (FA-mBRNP@C3G-Fe³⁺) without NIR irradiation; FIG. 11G shows a detection result of experimental group (FA-mBRNP@C3G-Fe³⁺) with NIR irradiation; and FIG. 11H shows a detection result in bright field of experimental group (FA-mBRNP@C3G-Fe3+) with NIR irradiation.

FIG. 12A to FIG. 12J show test results of intracellular ROS after macrophage M1 polarization induced by lipopolysaccharide (LPS) stimulation, in which, FIG. 12A shows a test result of intracellular ROS of blank control group; FIG. 12B shows a test result of intracellular ROS with LPS stimulation; FIG. 12C shows a test result of intracellular ROS after adding BRNP; FIG. 12D shows a test result of intracellular ROS after adding mBRNP; FIG. 12E shows a test result of intracellular ROS after adding FA-mBRNP@C3G-Fe³⁺; FIG. 12F shows shows a test result in bright field of blank control group; FIG. 12G shows a test result in bright field with LPS stimulation; FIG. 12H shows a test result in bright field after adding BRNP; FIG. 12I shows a test result in bright field after adding mBRNP; FIG. 12J shows a test result in bright field after adding FA-mBRNP@C3G-Fe³⁺.

FIG. 13 shows expression results of M1 marker IL-13.

FIG. 14 shows expression results of M2 marker IL-10.

FIG. 15 shows a microscopic appearance of microneedle (MN).

FIG. 16 shows test results of the tissue penetration of MN.

FIG. 17 shows test results of the mechanical strength of MN.

FIG. 18A to FIG. 18C show co-localization observation results of FA-mBRNP@C3G-Fe³⁺ in MN, in which, FIG. 18B shows the bright field image of MN; FIG. 18C shows fluorescent image of FA-mBRNP@C3G-Fe³⁺ in MN; and FIG. 18A shows merged results of FIG. 18B and FIG. 18C.

FIG. 19 shows sustained release results of FA-mBRNP@C3G-Fe³⁺ in MN.

FIG. 20A to FIG. 20L show the alveolar bone resorption and treatment effects in each group, in which, FIG. 20A shows a stereomicroscope image of the control group; FIG. 20B shows a stereomicroscope image of the periodontitis group; FIG. 20C shows a stereomicroscope image of the microneedle treatment group; and FIG. 20D shows a stereomicroscope image of the microneedle photothermal treatment group; FIG. 20E shows a micro-CT imaging of the control group; FIG. 20F shows a micro-CT imaging of the periodontitis group; FIG. 20G shows a micro-CT imaging of the microneedle treatment group; FIG. 20H shows a micro-CT imaging of the microneedle photothermal treatment group; FIG. 20I shows the control group observed by biological microscope after being stained with H&E; FIG. 20J shows the periodontitis group observed by biological microscope after being stained with H&E; FIG. 20K shows the microneedle treatment group observed by biological microscope after being stained with H&E; and FIG. 20L shows the microneedle photothermal treatment group observed by biological microscope after being stained with H&E.

### DETAILED DESCRIPTION OF THE EMBODIMENTS

The present disclosure provides a near-infrared responsive bilirubin composite nanoparticle, including a bilirubin-gelatin composite nanoparticle, wherein
a surface of the bilirubin-gelatin composite nanoparticle is coated with a macrophage cell membrane and an organic-metal coordination supramolecular network coating in sequence; and an outer surface of the near-infrared responsive bilirubin composite nanoparticle is modified with DSPE-PEG-FA;
in the bilirubin-gelatin composite nanoparticle, bilirubin and gelatin are covalently bonded via an amide bond; and
in the organic-metal coordination supramolecular network coating, an organic ligand is anthocyanin, and a coordinating metal ion is a ferric ion.

In some embodiments of the present disclosure, the bilirubin-gelatin composite nanoparticle has a particle size of 100 nm to 300 nm, and preferably 200 nm to 300 nm. In some embodiments, the near-infrared responsive bilirubin composite nanoparticle has a particle size of 100 nm to 500 nm, and preferably 200 nm to 400 nm.

In the present disclosure, in the organic-metal coordination supramolecular network coating, bilirubin and gelatin are covalently bonded via an amide bond. In some embodiments, a mass ratio of the bilirubin to the gelatin is 1:10.

In the present disclosure, a method for preparing the near-infrared responsive bilirubin composite nanoparticle includes the following steps:
mixing the bilirubin, the gelatin, a carboxyl activator, and an organic solvent, and subjecting a resulting mixture to amidation reaction, to obtain a bilirubin-gelatin composite;
mixing an aqueous dispersion of the bilirubin-gelatin composite with an organic solvent, and subjecting a resulting mixture to self-assembly, to obtain a bilirubin-gelatin composite nanoparticle;
subjecting the bilirubin-gelatin composite nanoparticle and the macrophage cell membrane to co-extrusion, to obtain a cell membrane-coated bilirubin-gelatin composite nanoparticle;
mixing an aqueous dispersion of the cell membrane-coated bilirubin-gelatin composite nanoparticle, the anthocyanin, a soluble ferric ion source, and water, and adjusting a pH value of a resulting mixed solution to 7.5 to 8, to obtain an organic-metal coordination supramolecular network coating-coated bilirubin-gelatin composite nanoparticle; and
mixing an aqueous dispersion of the organic-metal coordination supramolecular network coating-coated bilirubin-gelatin composite nanoparticle with the DSPE-PEG-FA, and subjecting a resulting mixture to incubation to obtain the near-infrared responsive bilirubin composite nanoparticle.

In the present disclosure, the bilirubin, the gelatin, a carboxyl activator, and an organic solvent are mixed, and a resulting mixture is subjected to amidation reaction to obtain a bilirubin-gelatin composite. In some embodiments, the carboxyl activator is a combination of N-hydroxysuccinimide (NHS) and 1-ethyl-3-(3-dimethylaminopropyl)carbodiimide hydrochloride (EDC). In some embodiments, a mass ratio of the bilirubin to the gelatin is in a range of 1:(10-15), and preferably 1:(12-14). In some embodiments, a molar ratio of the bilirubin to the NHS is in a range of 1:(1-2), preferably 1:1. In some embodiments, a molar ratio of the bilirubin to the EDC is in a range of 2:(7-14), and preferably 2:(10-12).

In some embodiments of the present disclosure, the organic solvent is dimethyl sulfoxide (DMSO).

In some embodiments of the present disclosure, the mixing is performed as follows: mixing the bilirubin, the carboxyl activator, and the organic solvent by stirring, and then adding the gelatin thereto. In some embodiments, the mixing by stirring is conducted for 30 min.

In some embodiments of the present disclosure, the amidation reaction is conducted in an anaerobic environment. In some embodiments, the amidation reaction is conducted at room temperature for 8 h to 10 h, and preferably 9 h.

In the present disclosure, after the amidation reaction, a resulting amidation reaction solution is subjected to dialysis and drying to obtain a bilirubin-gelatin composite solid. In some embodiments, the dialysis includes first dialysis and second dialysis in sequence; In some embodiments, the first dialysis is conducted in a dialysate of 10 mM sodium hydroxide solution for 4 h to 8 h, and preferably 5 h to 6 h. In some embodiments, the second dialysis is conducted in a dialysate of water for preferably 24 h to 36 h.

In some embodiments of the present disclosure, the drying is performed by freeze-drying. There are no special requirements for freeze-drying means, and freeze-drying means well known to those skilled in the art may be used.

In the present disclosure, an aqueous dispersion of the bilirubin-gelatin composite is mixed with an organic solvent, and a resulting mixture is subjected to self-assembly to obtain a bilirubin-gelatin composite nanoparticle. In some embodiments, the aqueous dispersion of the bilirubin-gelatin composite has a bilirubin-gelatin composite concentration of 1 mg/mL to 5 mg/mL, and preferably 2 mg/mL to 4 mg/mL. In some embodiments, the organic solvent is chloroform. In some embodiments, the mixing is performed by stirring. In some embodiments, the mixing by stirring is conducted for 15 min.

In some embodiments of the present disclosure, the self-assembly is conducted under an ultrasonic condition. In some embodiments, the ultrasonic for providing the ultrasonic condition has a power of 100 W to 150 W and a frequency of 40 kHz to 60 kHz, and preferably 50 kHz; and the ultrasonic condition is maintained for 20 min to 30 min, and preferably 25 min.

In some embodiments of the present disclosure, after the self-assembly, an obtained solution is subjected to solid-liquid separation. In some embodiments, the solid-liquid separation is conducted by centrifugation. In some embodiments, the centrifugation is conducted at 12,000 rpm to 14,000 rpm for 8 min to 12 min, preferably 10 min.

In the present disclosure, the bilirubin-gelatin composite nanoparticle and the macrophage cell membrane are subjected to co-extrusion to obtain a cell membrane-coated bilirubin-gelatin composite nanoparticle. In some embodiments of the present disclosure, the macrophage cell membrane is a cell membrane of a mouse macrophage cell line Raw 264.7. As a specific example, the macrophage cell membrane is extracted according to the instructions of a membrane protein extraction kit provided by Beyotime Biotech. Inc., China.

In some embodiments of the present disclosure, a mass ratio of the bilirubin-gelatin composite nanoparticle to the macrophage cell membrane is in range of (1-2):1. In some embodiments, the co-extrusion is conducted using a micro-extruder.

In some embodiments of the present disclosure, after the co-extrusion, a resulting co-extrusion product is centrifuged at a centrifugal force of 12,000 g for 10 min to 15 min, and preferably 12 min to 14 min.

In the present disclosure, an aqueous dispersion of the cell membrane-coated bilirubin-gelatin composite nanoparticle, the anthocyanin, a soluble ferric ion source, and water are mixed, and a pH value of a resulting mixed solution is adjusted to 7.5 to 8 to obtain an organic-metal coordination supramolecular network coating-coated bilirubin-gelatin composite nanoparticle. In some embodiments, the aqueous dispersion of the cell membrane-coated bilirubin-gelatin composite nanoparticle has a concentration of 50 μg/mL.

In some embodiments of the present disclosure, the soluble ferric ion source is ferric chloride, and preferably ferric chloride hexahydrate. In some embodiments, a mass ratio of the anthocyanin, the soluble ferric ion source, and the cell membrane-coated bilirubin-gelatin composite nanoparticle is in a range of 5:1:(4-5).

In some embodiments of the present disclosure, the pH value of the mixed solution is adjusted to 7.5 to 8. In some embodiments, a pH adjuster used to adjust the pH value is as follows: dilute hydrochloric acid and a sodium hydroxide solution.

In some embodiments of the present disclosure, the anthocyanin and the ferric ion are subjected to coordination reaction at room temperature for 3 min to 5 min, and preferably 4 min after the pH value of the mixed solution is adjusted to 7.5 to 8.

In some embodiments of the present disclosure, after the coordination reaction, a resulting coordination reaction solution is centrifuged at a centrifugal force of 12,000 g for 10 min to 15 min, and preferably 12 min to 14 min.

In the present disclosure, an aqueous dispersion of the organic-metal coordination supramolecular network coating-coated bilirubin-gelatin composite nanoparticle is mixed with the DSPE-PEG-FA, and a resulting mixture is subjected to incubation, to obtain the near-infrared responsive bilirubin composite nanoparticle. In some embodiments, the aqueous dispersion of the organic-metal coordination supramolecular network coating-coated bilirubin-gelatin composite nanoparticle has a concentration of 200 μg/mL.

In some embodiments of the present disclosure, a mass ratio of the organic-metal coordination supramolecular network coating-coated bilirubin-gelatin composite nanoparticle to the DSPE-PEG-FA is in a range of (4-10):1. In some embodiments, the incubation is conducted at a temperature of 35° C. to 37° C. for 0.5 h to 2 h, and preferably 1 h to 1.5 h. In some embodiments of the present disclosure, after the incubation, a resulting mixture is centrifuged at a centrifugal froce of 12,000 g for 10 min to 15 min, and preferably 12 min to 14 min.

The present disclosure further provides use of the near-infrared responsive bilirubin composite nanoparticle in preparation of a drug for treating periodontitis.

The present disclosure further provides a near-infrared responsive bilirubin composite nanoparticle-loaded microneedle for periodontitis, including a microneedle tip and a microneedle base, wherein raw materials for preparing the microneedle tip include GelMA, a photoinitiator, and the near-infrared responsive bilirubin composite nanoparticle.

In some embodiments of the present disclosure, the photoinitiator is lithium phenyl (2,4,6-trimethylbenzoyl) phosphinate (LAP). In some embodiments, a mass ratio of the GelMA, the photoinitiator, and the near-infrared responsive bilirubin composite nanoparticle is a range of 200: 5:(8-10).

In some embodiments of the present disclosure, a raw material for preparing the microneedle base includes gelatin. In some embodiments, the gelatin is provided in the form of an aqueous gelatin solution. In some embodiments, the aqueous gelatin solution has a concentration of 10 wt %.

In the present disclosure, a method for preparing the near-infrared responsive bilirubin composite nanoparticle-loaded microneedle for periodontitis includes the following steps:
mixing the GelMA, the photoinitiator, and the near-infrared responsive bilirubin composite nanoparticle to obtain a mixture; and
adding the mixture into a negative mold of the drug loading microneedle, spreading the gelatin flatly on the negative mold, and conducting ultraviolet (UV) curing to obtain the near-infrared responsive bilirubin composite nanoparticle-loaded microneedle for periodontitis.

In some embodiments of the present disclosure, the UV light used for UV curing has a wavelength of 360 nm to 480 nm and an intensity of 50 W/cm², and the UV curing is conducted for 3 min to 5 min, and preferably 4 min.

In some embodiments of the present disclosure, after the UV curing, a resulting cured product is left to stand in the dark at room temperature for 24 h.

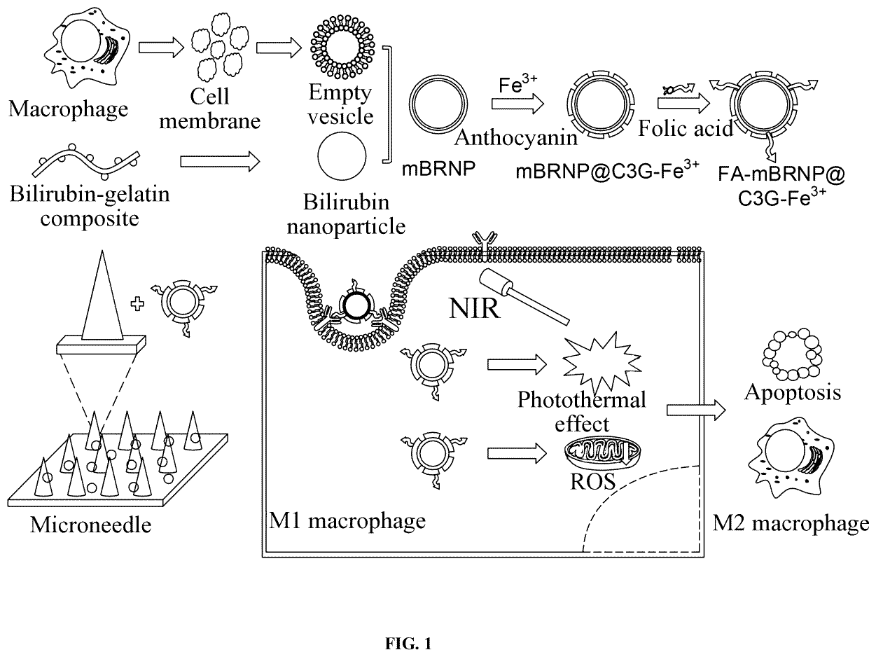

In the present disclosure, FIG. 1 shows flowcharts of the preparation of the near-infrared responsive bilirubin composite nanoparticle and a drug-loaded microneedle for periodontitis.

The near-infrared responsive bilirubin composite nanoparticle-loaded periodontal microneedle and the preparation method thereof according to the present disclosure will be described in detail in conjunction with the following examples, but they should not be construed as limiting the claimed scope of the present disclosure.

### Example 1

### (1) Preparation of a Bilirubin-Gelatin Composite (BR-GA)

20 μmol of BR, 20 μmol of NHS, and 70 μmol of EDC were added into 4 mL of DMSO, and a resulting mixture was stirred at room temperature for 30 min. 100 mg of GA was added thereto, and a resulting mixture was subjected to incubation in an anaerobic incubator overnight. A resulting mixture was placed in 6 mL of a 10 mM sodium hydroxide solution, and dialysed with the 10 mM sodium hydroxide solution for 4 h, followed by continued dialysis with distilled water for 24 h, and then freeze-drying, to obtain the BR-GA.

### (2) Preparation of a Bilirubin-Gelatin Composite Nanoparticle (BRNP)

5 mg of the BR-GA was dissolved in 1 mL of distilled water, and 0.25 mL of chloroform was slowly added dropwise. A resulting solution was stirred at room temperature for 15 min and then sonicated (at 40 kHz, 100 W) for 30 min. A supernatant was collected and centrifuged (12,000 rpm, 10 min) to obtain a precipitate, which was the BRNP.

(3) Preparation of a Cell Membrane-Coated Bilirubin-Gelatin Composite Nanoparticle (mBRNP)

The cell membrane of a mouse macrophage cell line Raw 264.7 was extracted according to the instructions of a membrane protein extraction kit provided by Beyotime Biotech. Inc., China. The cell membrane and BRNP were co-extruded through a micro-extruder. Gradient centrifugation was conducted at a centrifugal force of 3,000 g for 30 min and then at a centrifugal force of 12,000 g for 30 min, to obtain a precipitate, which was the mBRNP.

(4) Preparation of an Organic-Metal Coordination Supramolecular Network Coating-Coated Bilirubin-Gelatin Composite Nanoparticle (mBRNP@C3G-Fe³⁺ )

A mixed solution was prepared with concentrations as follows: 0.04 mg/ml C3G; 0.008 mg/mL ferric chloride hexahydrate; and 50 μg mBRNP. The pH value of the mixed solution was adjusted to 8.0. A supernatant was collected and centrifuged (12,000 rpm, 10 min) to obtain the mBRNP@C3G-Fe³⁺.

(5) Preparation of a near-infrared responsive bilirubin composite nanoparticle (FA-mBRNP@C3G-Fe³⁺)

1 mL 200 μg mBRNP@C3G-Fe³⁺ was co-incubated with 50 μg DSPE-PEG-FA at 37° C. for 1 h, and a resulting mixture was then centrifuged (10,000 g, 10 min). A precipitate was collected, which was the FA-mBRNP@C3G-Fe³⁺.

### Structural Characterization

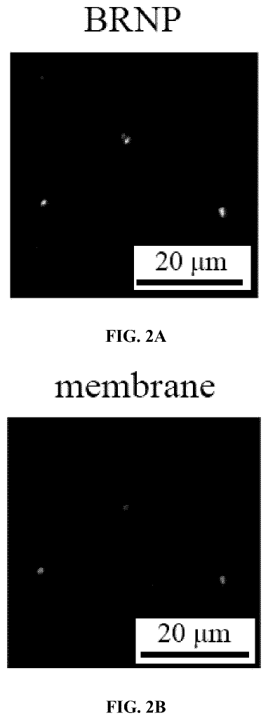

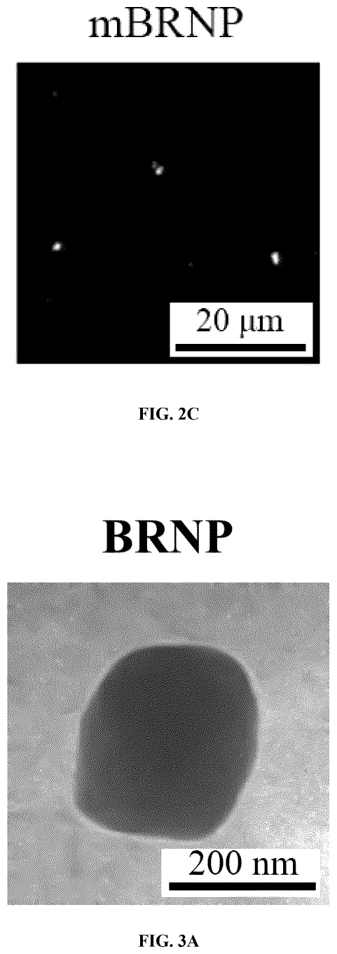

FIG. 2A and FIG. 2C show fluorescence co-localization images of BRNP and mBRNP, respectively. Nanoparticles were successfully coated with cell membrane, and macrophage cell membrane (exhibiting red fluorescence, as shown in FIG. 2B) and BRNP (exhibiting green fluorescence, as shown in FIG. 2A) were well co-localized.

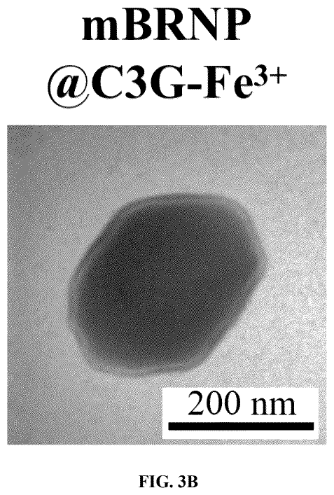

FIG. 3A and FIG. 3B show TEM images of BRNP and mBRNP@C3G-Fe³⁺. As can be seen, the BRNP is in the shape of a regular round and has a particle size of approximately 200 nm. A composite coating is formed on a surface of the mBRNP@C3G-Fe³⁺, with an obvious core-shell structure, a slightly increased particle size and clearer surface boundaries.

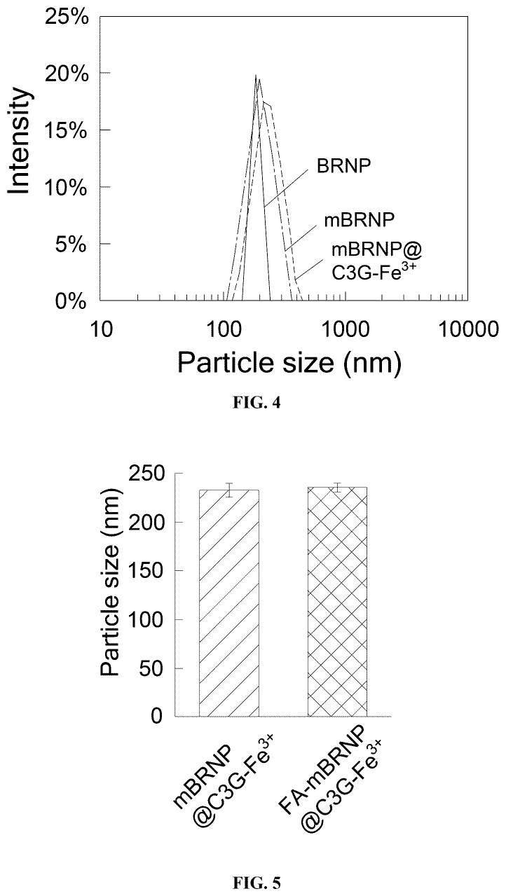

FIG. 4 shows particle size distributions of BRNP, mBRNP, and mBRNP@C3G-Fe³⁺. The particle size distribution results show that the particle size of the prepared BRNP is mainly concentrated around 200 nm. After being coated with macrophage cell membrane (mBRNP) and organic-metal supramolecular network coating (mBRNP@C3G-Fe³⁺), the particle size of the material increases slightly, proving the successful biomodification of the material.

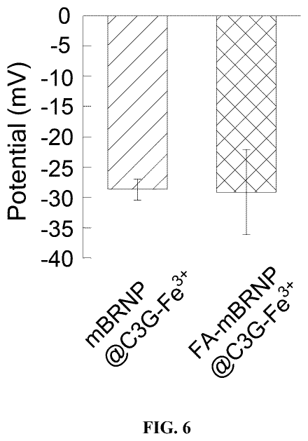

Particle sizes of the mBRNP@C3G-Fe³⁺ and FA-mBRNP@C3G-Fe³⁺ are shown in FIG. 5 and potentials thereof are shown in FIG. 6. Particle size analysis shows that FA modification does not affect the size of the nanoparticles, and an average particle size is approximately 230 nm. Potential analysis results show that the potential of the nanoparticles is about −27 mV, which is close to a cell membrane potential, confirming that the FA modification does not damage the cell membrane structure on the surface of the nanoparticles.

### Performance Testing

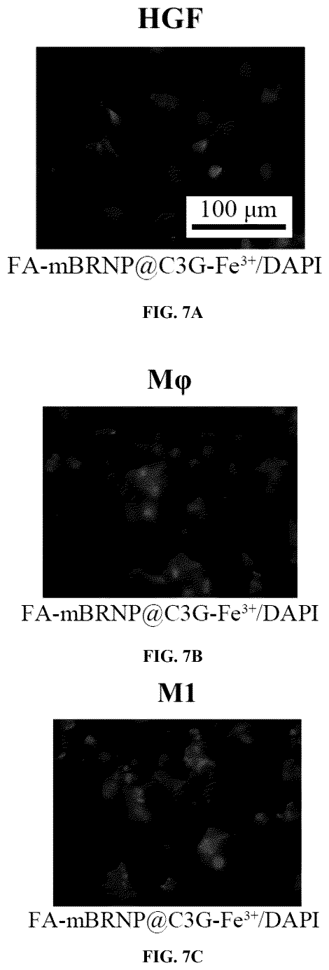

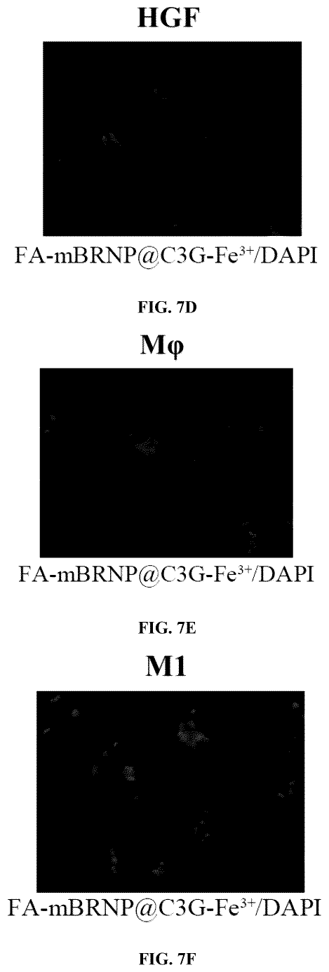

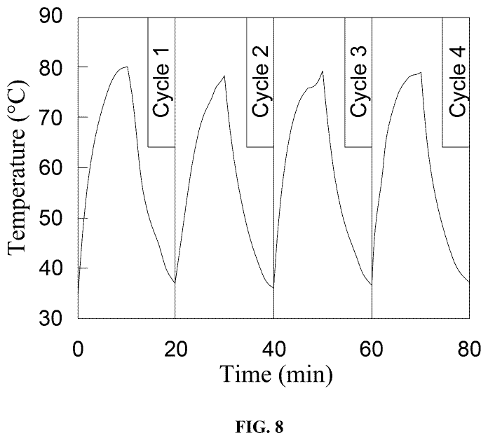

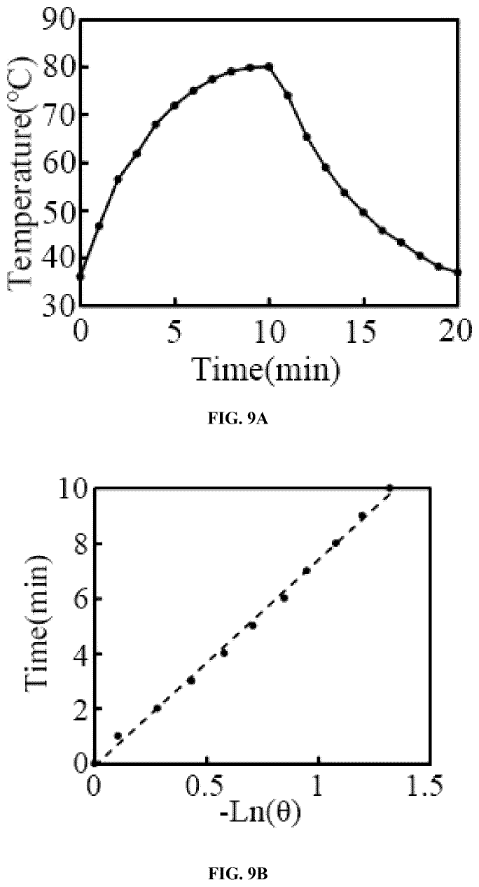

(1) In order to prove the ability of M1 macrophages to specifically uptake FA-mBRNP@C3G-Fe³⁺ in vitro, fibroblasts and M_{φ} macrophages were used as control groups and M1 macrophages were used as experimental groups to determine the ability of different cells in nanoparticle uptake. The MI polarization of macrophages was pre-induced with 1 μg/mL lipopolysaccharide (LPS) for 24 h as experimental groups. 10 μM of FA-mBRNP@C3G-Fe³⁺ nanomaterial labeled with red fluorescent Dil was added into fibroblasts, M_{φ} macrophages, and M1 macrophages separately, and they were co-cultured in a 37° C. incubator for 8 h, and the unphagocytosed FA-mBRNP@C3G-Fe³⁺ was washed off. The above cells were fixated with 4% paraformaldehyde solution for 10 min, their nuclei were stained with DAPI, and then observed under a fluorescence microscope. The results are shown in FIG. 7A to FIG. 7F. Compared with fibroblasts and M_{φ} macrophages, the phagocytosis level of FA-mBRNP@C3G-Fe³⁺ by M1 macrophages is significantly increased, indicating that FA-mBRNP@C3G-Fe³⁺ shows a desirable targeting ability to M1 macrophages.
(2) The mBRNP@C3G-Fe 3+with a concentration of 80 μM was placed in an EP tube, and then irradiated with NIR at a power of 1.5 W cm⁻² for 10 min, and then naturally cooled to 37° C. after irradiation. The above process was repeated for 4 cycles, photothermal images were collected using an infrared thermal imager and temperature changes were recorded. The temperature-time change curves are shown in FIG. 8, and the photothermal conversion efficiency is shown in FIG. 9A and FIG. 9B.

As shown in FIG. 8, under 808 nm NIR irradiation, the mBRNP@C3G-Fe³⁺ generates considerable heat, causing the temperature to rapidly increase to approximately 80° C. within 10 min, and maintains a relatively stable and repeatable heating-cooling process in 4 cycles, indicating that mBRNP@C3G-Fe³⁺ has a stable photothermal conversion ability. As shown in FIG. 9A and FIG. 9B, a heat transfer time constant calculated through a single cycle is 448.63. This indicates that the mBRNP@C3G-Fe³⁺ has excellent photothermal conversion performance and significant photothermal stability, and is an effective photothermal agent.

(3) In the present disclosure, the photothermal ability of FA-mBRNP@C3G-Fe³⁺ to induce M1 macrophage apoptosis was evaluated. A blank control group (Control) without medication and an experimental group with medication (FA-mBRNP@C3G-Fe³⁺) were set up. Under the same conditions, NIR irradiation treatment was not performed/performed (NIR parameters: 808 nm, 1.5 W cm₋₂). Subsequently, cell viability of the M1 macrophage in each group was evaluated using CCK-8.

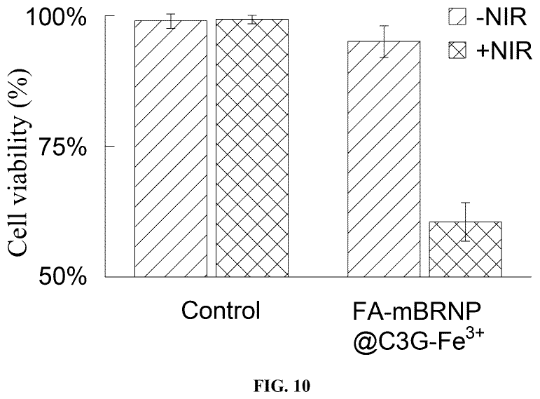

FIG. 10 shows photothermal-induced apoptosis of M1 macrophages. The control group has no significant effect on cell viability of M1 macrophage in regardless of with or without NIR irradiation; after adding FA-mBRNP@C3G-Fe³⁺, there is still no obvious effect on cell viability without NIR irradiation. This proves that the prepared nanoparticles have no obvious toxic side effects on cells and shows desirable biocompatibility. In the FA-mBRNP@C3G-Fe³⁺ group, the cell viability decreases by about 40% after NIR irradiation stimulation, indicating that M1 macrophages after ingesting the FA-mBRNP@C3G-Fe³⁺ could be stimulated by NIR irradiation to reduce cell viability.

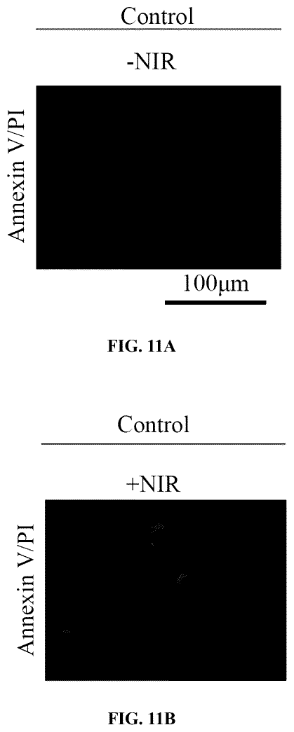

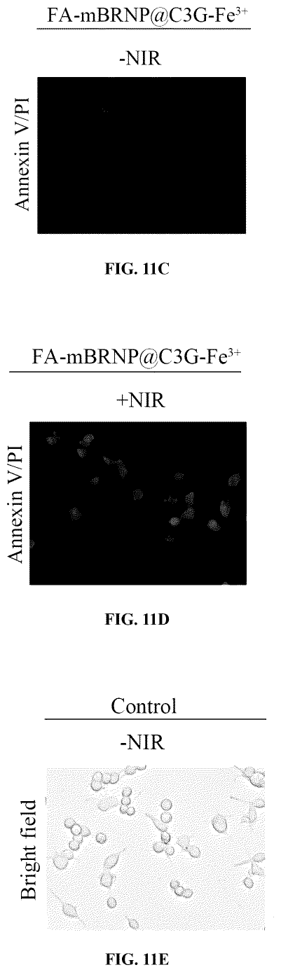

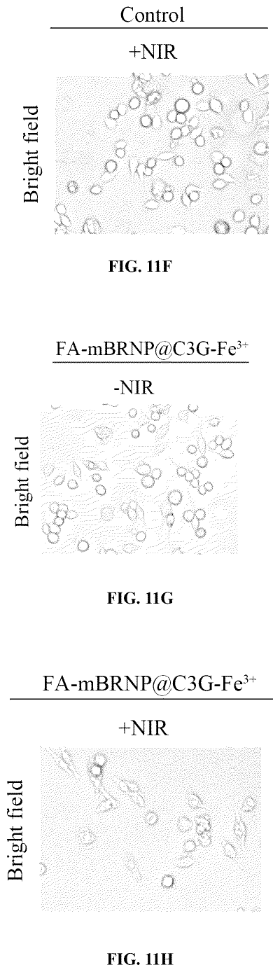

Further, the cells were stained and observed using Annexin V-FITC apoptosis detection kit. FIG. 11A to FIG. 11H show detection results of apoptosis using Annexin V-FITC. In the FA-mBRNP@C3G-Fe³⁺ experimental groups, a large number of cells express the apoptosis marker Annexin V at a high level with their cell membranes intact after NIR irradiation, confirming that the FA-mBRNP@C3G-Fe³⁺ nanomaterial could undergo photothermal conversion and successfully induce apoptosis of M1 macrophages.

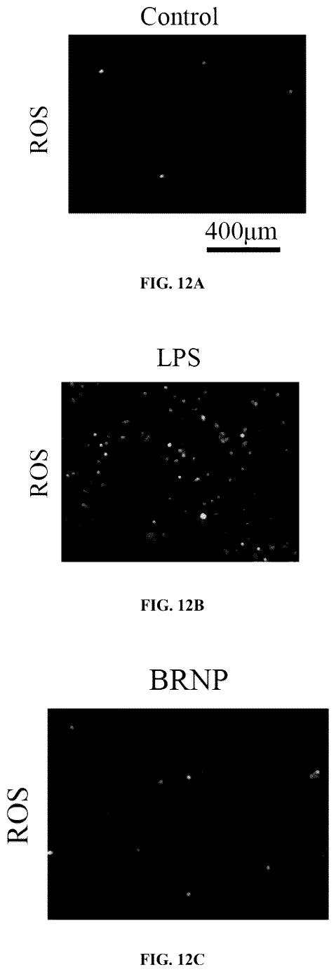

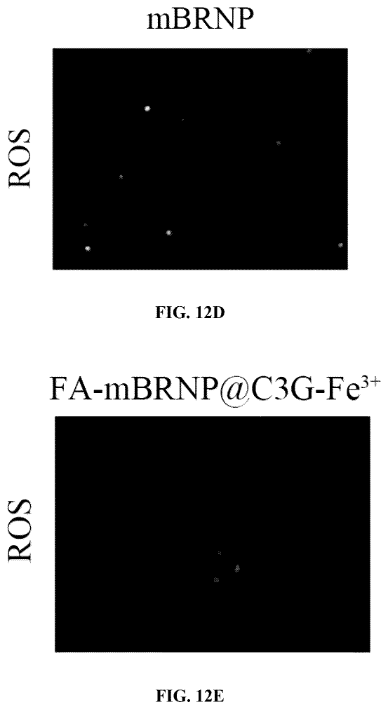

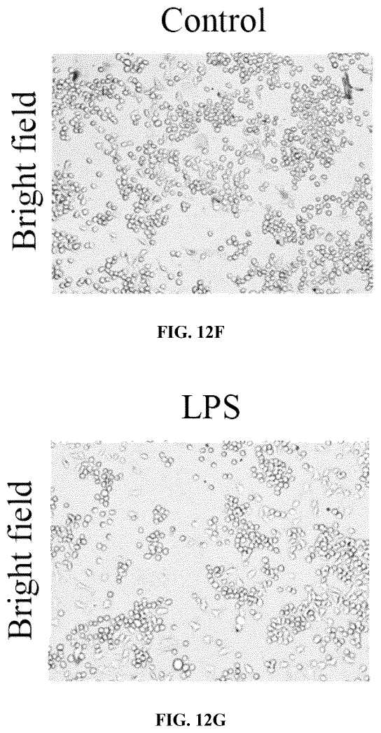

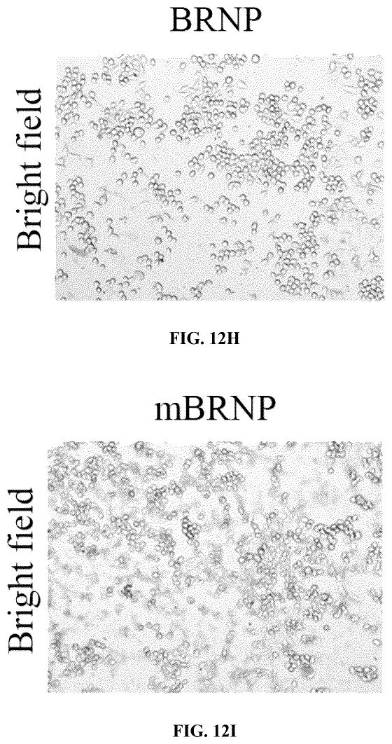

(4) After the mouse macrophage cell line RAW264.7 was stimulated with 1 μg/mL LPS to induce M1 polarization, BRNP, mBRNP, and mBRNP@C3G-Fe³⁺ were separately added thereto, and they were then incubated in a cell incubator for 24 h separately. The cells were detected using Beyotime's cellular reactive oxygen species detection kit, stained with 10 μM DCFH-DA reactive oxygen specie (ROS) fluorescent probe according to the instructions, and then observed using a fluorescence microscope. The results are shown in FIG. 12A to FIG. 12J. The results show that intracellular ROS significantly increases after LPS stimulation-induced macrophage M1 polarization; the addition of BRNP, mBRNP, and mBRNP@C3G-Fe³⁺ could reduce intracellular ROS to a certain extent. Among them, the ROS in the cells treated with mBRNP@C3G-Fe³⁺ could be restored to close to normal levels. This indicates that the near-infrared responsive bilirubin composite nanoparticle exhibits an excellent antioxidant capacity.

(5) The M2 polarization effect of the near-infrared responsive bilirubin composite nanoparticle prepared in the present disclosure on macrophages was detected by PCR. The mouse macrophage cell line RAW264.7 was stimulated with 1 μg/mL LPS to simulate an inflammatory state to induce M1 polarization, and BRNP, mBRNP, mBRNP@C3G-Fe³⁺, and FA-mBRNP@C3G-Fe³⁺ were added separately thereto, and culture was continued for 24 h, and cells in each group were collected for PCR detection.

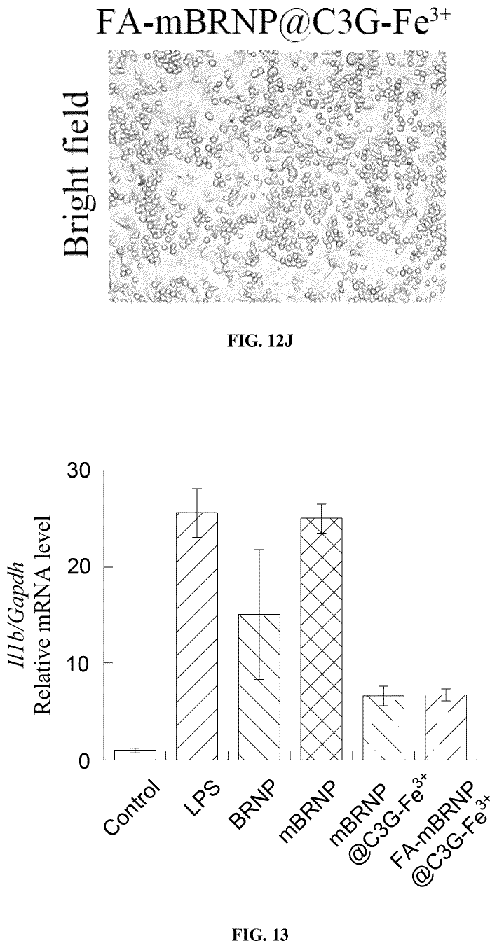

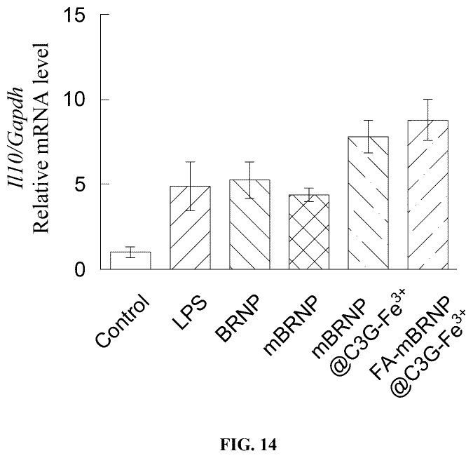

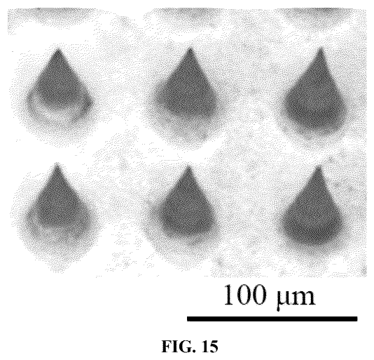

The expression results of the M1 marker IL-1β are shown in FIG. 13, while the expression results of the M2 marker IL-10 are shown in FIG. 14. An approximately 26-fold increase in the expression level of the M1 marker IL-1β is observed in the LPS group, while the expression level of IL-1β is significantly reduced after nanoparticle treatment. It is also observed that the expression level of M2 marker IL-10 increases significantly after treatment with FA-mBRNP@C3G-Fe³⁺, indicating that FA-mBRNP@C3G-Fe³⁺ could promote the M2 repolarization.

### Example 2

Preparation of a microneedle (MN) for periodontitis

A photoinitiator and the FA-mBRNP@C3G-Fe³⁺ were added into GelMA, wherein a mass ratio of the GelMA, the photoinitiator, and the FA-mBRNP@C3G-Fe³⁺ was 200:5:8, after thoroughly mixing, a resulting mixture was poured into a negative mold of an MN to form an MN body. An aqueous GA solution with a concentration of 10 wt % was evenly spread thereon to form an MN base. It was then irradiated under UV (wavelength: 360-480 nm, intensity: 50 W/cm²) for 5 min. After being placed in the dark at room temperature for 24 h, a drug-loading MN was obtained by demolding.

A microscopic appearance of the MN was observed using a stereomicroscope. The results are shown in FIG. 15. The MNs are conical in shape, uniformly loaded, and regularly arranged.

### Performance Testing

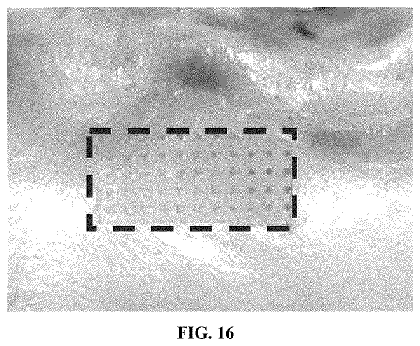

(1) The mechanical properties of microneedles are key factors in whether they could penetrate periodontal tissues. Pig jaw bones were used to simulate the in vivo application scenario of MN to test the tissue penetration of MN. The results are shown in FIG. 16. The results show that the MN could successfully penetrate the pig gingival tissue (shown in the black box).

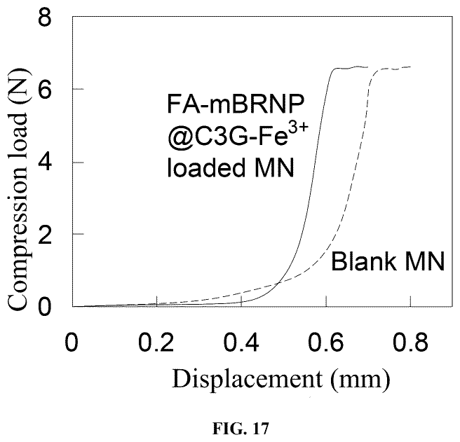

A mechanical strength of the MN was further tested through a compression test. The tip of MN was placed vertically on a stainless steel plate and pressed against another stainless steel plate at a constant speed of 0.5 mm/min. A relationship between the applied pressure and the deformation of the MN was recorded. The results are shown in FIG. 17. The mechanical strength of the microneedle with the FA-mBRNP@C3G-Fe3+is slightly reduced and has sufficient mechanical strength to penetrate freshly extracted porcine gingival tissue.

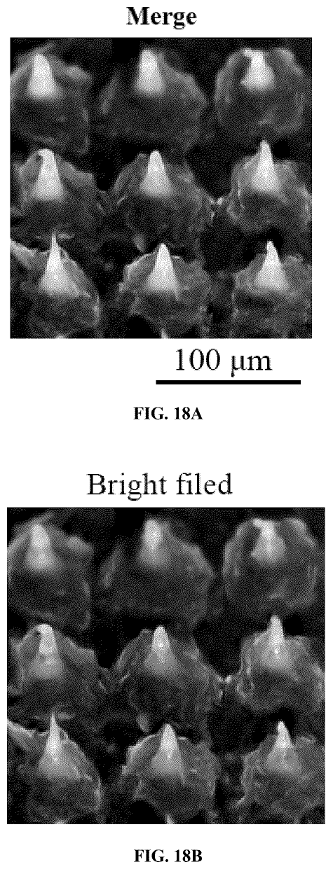

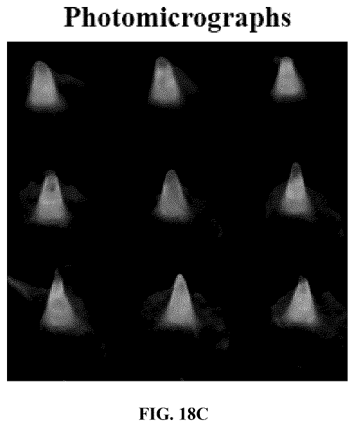

(2) Co-localization observation of FA-mBRNP@C3G-Fe³⁺ in MN was conducted using fluorescence microscope. The results are shown in FIG. 18A to FIG. 18C. The fluorescence images show that the FA-mBRNP@C3G-Fe³⁺ particles are evenly distributed in the tip.

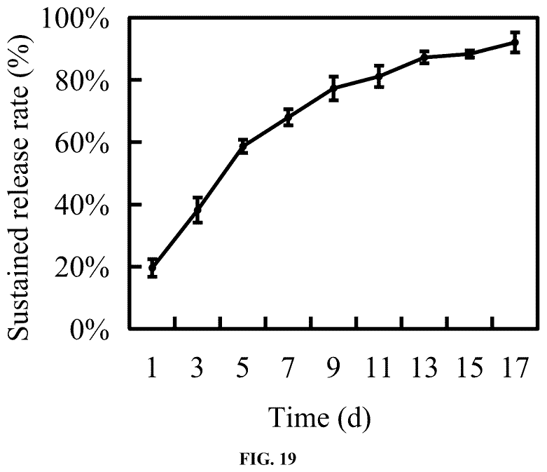

Subsequently, the microneedles loaded with FA-mBRNP@C3G-Fe³⁺ were placed in 1 mL of PBS, and the in vitro sustained release performance was evaluated at a constant temperature of 37° C. The sample supernatants were collected at different time points (1, 3, 5, 7, 9, 11, 13, 15, 17 days), the absorbance was measured at a wavelength of 450 nm using a UV-visible spectrophotometer, and a release amount of FA-mBRNP@C3G-Fe³⁺ was calculated. FIG. 19 shows sustained release results of FA-mBRNP@C3G-Fe³⁺. The sustained release curve shows that the nanoparticles were largely released within 1 week, approximately 70%, and slowly and continuously released for up to two weeks, indicating a desirable sustained release performance.

(3) The mice were randomly divided into 4 groups: a control group, a periodontitis group, a microneedle treatment group, and a microneedle photothermal treatment group. A mouse periodontitis model was established according to standards. After the model was successfully established, the microneedle was separately inserted into the palatal gums of the mice in the microneedle treatment group and the microneedle photothermal treatment group. 1 day later, the microneedle photothermal treatment group was subjected to NIR irradiation for 5 min. After 7 days of treatment, the mice were euthanized, and their jaw alveolar bone specimens were collected, which were photographed using a stereomicroscope and scanned by micro-CT. Finally, the decalcified specimens were embedded with paraffin and then stained with hematoxylin and eosin (H&E). The alveolar bone resorption and treatment effect in each group were evaluated.

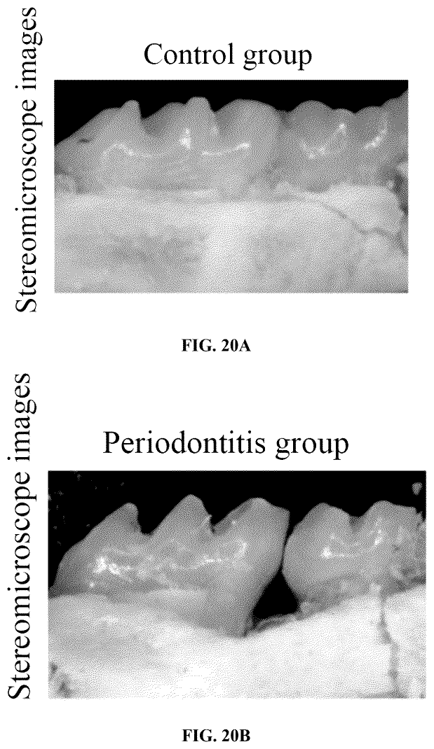

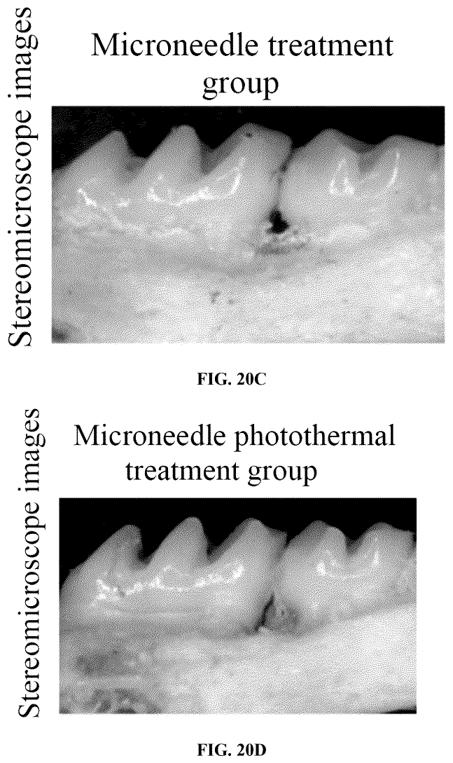

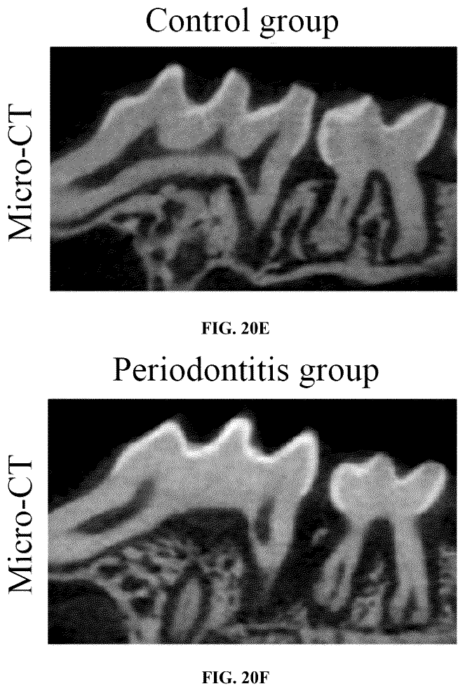

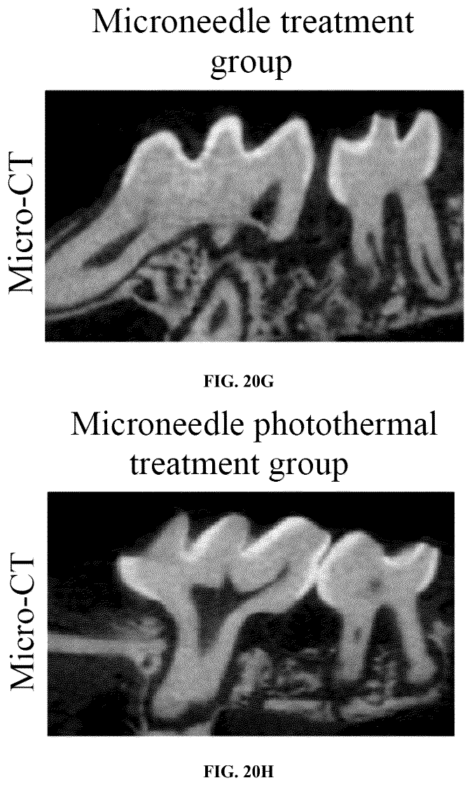

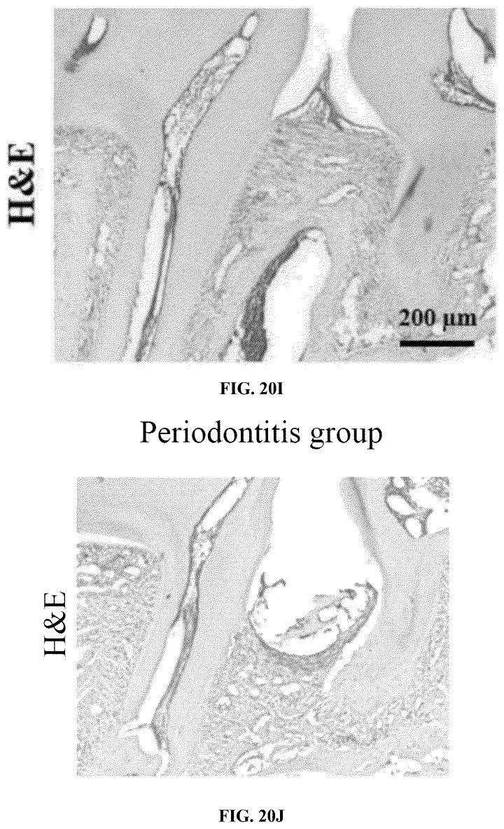

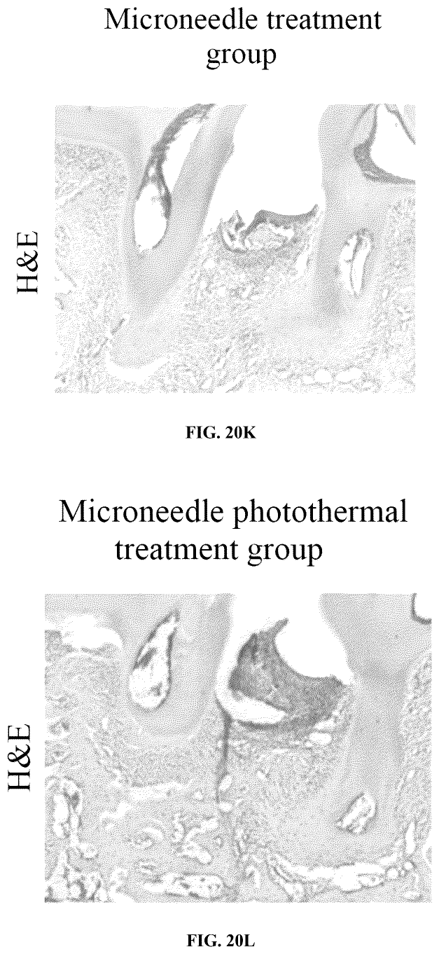

FIG. 20A to FIG. 20L shows the alveolar bone resorption and treatment effects in each group. Taking the control group as a reference, the alveolar bone resorption in the periodontitis group is the highest; the alveolar bone resorption in the microneedle treatment group is partially improved; and the alveolar bone resorption in the microneedle photothermal treatment group is the least, closest to the control group. This indicates that the near-infrared-responsive bilirubin composite nanoparticle-loaded microneedle prepared in the present disclosure has a desirable therapeutic effect on periodontitis in mice, and the therapeutic effect could be further improved when combined with photothermal therapy.

The above are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

1. A near-infrared responsive bilirubin composite nanoparticle, comprising a bilirubin-gelatin composite nanoparticle, wherein
a surface of the bilirubin-gelatin composite nanoparticle is coated with a macrophage cell membrane and an organic-metal coordination supramolecular network coating in sequence;
an outer surface of the near-infrared responsive bilirubin composite nanoparticle is modified with 1,2-distearoyl-sn-glycero-3-phosphoethanolamine-N-[folate(polyethylene glycol)] (DSPE-PEG-FA);
in the bilirubin-gelatin composite nanoparticle, bilirubin and gelatin are covalently bonded via an amide bond; and
in the organic-metal coordination supramolecular network coating, an organic ligand is anthocyanin, and a coordinating metal ion is ferric ion.

2. The near-infrared responsive bilirubin composite nanoparticle as claimed in claim 1, wherein the bilirubin-gelatin composite nanoparticle has a particle size of 100 nm to 300 nm; and
the near-infrared responsive bilirubin composite nanoparticle has a particle size of 100 nm to 500 nm.

3. The near-infrared responsive bilirubin composite nanoparticle as claimed in claim 1, wherein a mass ratio of the bilirubin to the gelatin is in a range of 1:10 to 1:15.

4. A method for preparing the near-infrared responsive bilirubin composite nanoparticle as claimed in claim 1, comprising the steps of
mixing the bilirubin, the gelatin, a carboxyl activator, and an organic solvent, and subjecting a resulting mixture to amidation reaction, to obtain a bilirubin-gelatin composite;
mixing an aqueous dispersion of the bilirubin-gelatin composite with an organic solvent, and subjecting a resulting mixture to self-assembly, to obtain a bilirubin-gelatin composite nanoparticle;
subjecting the bilirubin-gelatin composite nanoparticle and the macrophage cell membrane to co-extrusion, to obtain a cell membrane-coated bilirubin-gelatin composite nanoparticle;
mixing an aqueous dispersion of the cell membrane-coated bilirubin-gelatin composite nanoparticle, the anthocyanin, a soluble ferric ion source, and water, and adjusting a pH value of a resulting mixed solution to 7.5 to 8, to obtain an organic-metal coordination supramolecular network coating-coated bilirubin-gelatin composite nanoparticle; and
mixing an aqueous dispersion of the organic-metal coordination supramolecular network coating-coated bilirubin-gelatin composite nanoparticle with the DSPE-PEG-FA, and subjecting a resulting mixture to incubation, to obtain the near-infrared responsive bilirubin composite nanoparticle.

5. The method as claimed in claim 4, wherein the carboxyl activator is a combination of N-hydroxysuccinimide (NHS) and 1-ethyl-3-(3-dimethylaminopropyl)carbodiimide hydrochloride (EDC).

6. The method as claimed in claim 5, wherein a molar ratio of the bilirubin to the NHS is in a range of 1:1 to 1:2; and a molar ratio of the bilirubin to the EDC is in a range of 2:7 to 2:14.

7. The method as claimed in claim 4, wherein a mass ratio of the bilirubin to the gelatin is in a range of 1:10 to 1:15;
the amidation reaction is conducted at room temperature for 8 h to 10 h; and
the amidation reaction is conducted in an anaerobic environment.

8. The method as claimed in claim 4, wherein the self-assembly is conducted under an ultrasonic condition; and
an ultrasonic for providing the ultrasonic condition has a power of 100 W to 150 W and a frequency of 40 kHz to 60 kHz, and the ultrasonic condition is maintained for 20 min to 30 min.

9. The method as claimed in claim 4, wherein a mass ratio of the bilirubin-gelatin composite nanoparticle to the macrophage cell membrane is in a range of 1:1 to 2:1.

10. The method as claimed in claim 9, wherein the macrophage cell membrane is a cell membrane of a mouse macrophage cell line Raw 264.7.

11. The method as claimed in claim 4, wherein a mass ratio of the anthocyanin, the soluble ferric ion source, and the cell membrane-coated bilirubin-gelatin composite nanoparticle is in a range of 5:1:(4-5).

12. The method as claimed in claim 4, further comprising subjecting the anthocyanin and the ferric ion to coordination reaction at room temperature for 3 min to 5 min after adjusting the pH value of the resulting mixed solution to 7.5 to 8.

13. The method as claimed in claim 4, wherein the aqueous dispersion of the organic-metal coordination supramolecular network coating-coated bilirubin-gelatin composite nanoparticle has a concentration of 200 μg/mL.

14. The method as claimed in claim 4, wherein a mass ratio of the organic-metal coordination supramolecular network coating-coated bilirubin-gelatin composite nanoparticle to the DSPE-PEG-FA is in a range of 4:1 to 10:1; and
the incubation is conducted at a temperature of 35° C. to 37° C. for 0.5 h to 2 h.

15. A method for treating periodontitis, comprises administrating the near-infrared responsive bilirubin composite nanoparticle as claimed in claim 1 to a subject in need thereof.

16. A near-infrared responsive bilirubin composite nanoparticle-loaded microneedle for periodontitis, comprising a microneedle tip and a microneedle base of a drug loading microneedle, wherein
raw materials for preparing the microneedle tip comprise gelatin methacryloyl (GelMA), a photoinitiator, and the near-infrared responsive bilirubin composite nanoparticle as claimed in claim 1.

17. The near-infrared responsive bilirubin composite nanoparticle-loaded microneedle for periodontitis as claimed in claim 16, wherein the photoinitiator is lithium phenyl (2,4,6-trimethylbenzoyl)phosphinate.

18. The near-infrared responsive bilirubin composite nanoparticle-loaded microneedle for periodontitis as claimed in claim 16, wherein a mass ratio of the GelMA, the photoinitiator, and the near-infrared responsive bilirubin composite nanoparticle is in a range of 200:5:(8-10).

19. A method for preparing the near-infrared responsive bilirubin composite nanoparticle-loaded microneedle for periodontitis as claimed in claim 16, comprising the steps of
mixing the GelMA, the photoinitiator, and the near-infrared responsive bilirubin composite nanoparticle to obtain a mixture; and
adding the mixture into a negative mold of the drug loading microneedle, spreading gelatin flatly on the negative mold, and conducting ultraviolet (UV) curing, to obtain the near-infrared responsive bilirubin composite nanoparticle-loaded microneedle for periodontitis.

20. The method as claimed in claim 19, wherein UV light used for the UV curing has a wavelength of 360 nm to 480 nm and an intensity of 50 W/cm²; and
the UV curing is conducted for 3 min to 5 min.
